(19) United States
(12) Patent Application Publication
(10) Pub. No.: US 2009/0299260 A1
(43) Pub. Date: Dec. 3, 2009

(54) TUBE FOR MEDICAL PURPOSES

(76) Inventors: Thomas Kreischer, Saarbrucken (DE); Uwe Ahr, Winterbach (DE); Wolfgang Schulz, St. Wendel (DE); Tobias Weber, St. Wendel (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/293,307

(22) PCT Filed: Apr. 5, 2007
(86) PCT No.: PCT/EP2007/003126

§ 371 Date: Jan. 27, 2009

(30) Foreign Application Priority Data
Apr. 6, 2006 (DE) 102006016300.1

(51) Int. Cl. A61M 39/00

(52) U.S. Cl. 604/4.01; 138/140

(57) ABSTRACT

The present invention relates to a PVC-free tube (100) for medical purposes comprising three layers (101, 102, 103) arranged one above another, wherein each of these layers contains a polyolefin, and wherein the inner layer (102) also contains at least 60% of a thermoplastic elastomer. The present invention further relates to a tube system comprising several tubes according to the invention, which are connected via connectors. The tube according to the invention makes it possible for the conveyance rate loss, for example in an extracorporeal blood circulation, to be less than 15%, preferably less than 10%.

The present invention relates to a tube for medical purposes and a tube system comprising a plurality of tubes according to the invention and the use of the tube or tube system according to the invention in an extracorporeal blood circulation.

Pump tubes, in particular in conjunction with peristaltic pumps, are used in the medical field for example to convey blood in extracorporeal blood circulations. It is necessary for example in a haemodialysis extracorporeal blood circulation to transport the blood at appropriate conveyance rates to give the patient an acceptable and brief period of treatment.

Such pump tubes are positioned, e.g. in roller pump systems as are customary in haemodialysis, around a rotor in a guide groove. The rotor presses one or more rollers onto the pump tube segment, with the result that the tube is compressed and occluded at this point. This occluded point is advanced by rotating the rotor along the tube and the liquid which is located in front of the roller in the direction of rotation is thus moved forward.

The mechanical material requirements to be met by such pump tubes are therefore very high. In particular, the tube or tube segment must have a high kink resistance in order that the tube does not already buckle when inserted into the circular guide as a result of the narrow bending radius, making it impossible to convey the liquid. The danger of buckling is still further increased by the pressure of the conveyance roller. This means that the tube must have a sufficiently flexible structure. This property is important in particular also in respect of the requirement that it must be possible to fully occlude the tube. It is also necessary for many applications to completely close the tube by appropriate tube clamps in order to completely interrupt the flow of liquid if required.

In addition, the elastic properties of the pump tubes are also of particular importance, for example for a smooth haemodialysis procedure. For a constant delivery rate, it is important that the tube returns as much as possible to its original shape after occlusion. Customary tubes made of polyolefins typically only achieve much poorer values.

It is furthermore necessary that a tube, in particular a pump tube within its abovementioned use in an extracorporeal blood circulation, can withstand the abrasive effects of the conveyance roller. This means that the outside of the tube must have a mechanical resistance high enough not to be destroyed by the friction and pressure of the rollers. Likewise, therefore, the inner side of the tube, which is in contact with the liquid to be transported, must have properties which do not adversely affect the conveyance process or contaminate the liquid.

In the case of tubes which consist of several layers or sheets of material, any abrasion of the layer material by the friction of the layers arranged one above another must also be avoided.

In addition, it must also be ensured that no disruptive noises occur during the pumping process due to sheets of material arranged in the inside rubbing against one another. It is imperative in particular to avoid such noises, which often occur periodically for example during a haemodialysis treatment, as they can have a significant psychological impact on the patient. It is therefore desirable that the inner layers, often pressed on top of one another, of the occluded tube, in the case of tubes consisting of several layers, exert only a slight friction on one another during the pumping process.

A further requirement when transporting biological liquids such as for example infusion solutions or blood through tubes is that such pump tubes must be sterilizable. The currently most widespread method for the sterilization of medical articles, in particular in respect of its simplicity of procedure and preservation of the required material quality, is heat sterilization. In this method, medical equipment, articles and solutions are sterilized by exposure to temperatures of approximately 121° C. or more, optionally also under excess pressure. A further requirement as regards the material quality of tubes is therefore also that the material used for the tube is not deformed by the heat sterilization and the mechanical properties (brittleness, kink resistance, restorability etc.) are not adversely affected.

A further requirement for the use of a tube, in particular as a pump tube, for example in an extracorporeal haemodialysis, is that its material quality should remain constant during the conveyance process. A loss of delivery rate of pump tubes while the pumping capacity remains constant is often to be observed in pump tubes. It is therefore desired that this loss of delivery rate in a pump be kept as small as possible. A customary value, for example when conveying blood in customary haemodialysis procedures, is a loss of approx. 20% of the delivery rate of pump tubes, which depends on the pump used, the dimension of the tube, the conveyance rate, etc. The conveyance rate loss of conventional PVC tubes at a customary conveyance rate of approx. 300 ml/min is approx. 13%. It is therefore also desirable to achieve an improvement in the loss of the delivery rate of the pump tubes, i.e. to still further reduce the conveyance rate loss.

The use of PVC (polyvinyl chloride) as starting material, in particular for pump tubes, already makes it possible to satisfy most of the above named requirements today. However, the disadvantage of PVC, which in itself is a brittle, hard material and is subject to thermal degradation, is that it can be used to produce medical films, tubes and similar only by using plasticizers.

However, the inevitably necessary use of plasticizers has the disadvantage that the requirement for biocompatibility of materials, in particular in disposable medical articles which come into contact with biological liquids, is not always satisfied in the case of PVC. Recent results suggest that common plasticizers used for PVC such as e.g. trimellitic acid ester or dioctyl phthalate are harmful to health. The liquids guided through tubes made of PVC material elute the plasticizers from the PVC and are thereby contaminated. This problem is therefore the subject of numerous studies. Efforts are therefore currently being made, in particular for the transport or storage of biological liquids, to avoid the use of PVC as contact material, because of the plasticizers which must necessarily be used.

Tubes for transporting biological liquids are usually used in tube systems, for example for extracorporeal blood circulations, whereby further requirements are placed on a tube, in particular in respect of its use as a pump tube, in particular through the connection of individual tube segments to other tube units or segments. The connections between the individual pump tube segments are often created by so-called connectors. These connectors preferably consist of easily processed chemically largely inert pre-shaped parts made of polypropylene (PP). For a secure connection of tube and connector, a laser welding process is preferably used in a customarily used production process. In general only thermodynamically compatible polymers can be welded in this way, with the result that the choice of material for pump tubes is therefore still further severely limited as a result of the preferred use of PP connectors.

U.S. Pat. No. 4,578,413 describes a medical tube which can also be used as pump tube. The material of this tube consists of a polymer composition made of a thermoplastic elastomer such as e.g. a hydrocarbon block copolymer, optionally with added polystyrene and polypropylene together with polysiloxanes with phenyl side chains. The tube consists of a single sheet of material. The disadvantage of using thermoplastic elastomers is avoided by using polysiloxanes. An elution of harmful substances upon contact with e.g. human blood is very possible through the further use of approx. 40% mineral oil. Furthermore, the polysiloxane used has a very great disadvantage in respect of industrial-scale marketing because of its extremely high price.

U.S. Pat. No. 4,613,640 describes a medical tube made of a polymer composition comprising a hydrocarbon block copolymer, such as for example SEBS or SBS, and a linear polysiloxane and also optionally polypropylene. In particular, it was an aim of this patent to enable the production of transparent medical articles such as e.g. tubes. Tubes consisting of several sheets of material are not mentioned.

U.S. Pat. No. 4,299,256 describes a tube, which can also be used as a pump tube, composed of a mixture of PVC and silicone oil. This mixture of PVC and silicone oil forms the material composition of the outer layer of the tube. The inner layer, which comes into contact with biological liquids, can be composed of polyolefins combined with undesired terephthalate plasticizers. There are no details of delivery rates and dimensions of the tube in this specification.

U.S. Pat. No. 6,187,400 describes a PVC-free tube with improved pumping properties. This tube has a multi-layered structure and is composed of polyethylene homo- and copolymers combined with polyalkyl esters and alkylene esters. This specification also refers in particular to the problem of using polyolefins in the production of medical tubes. The polyolefins used to date, in particular polypropylene and polyethylene, have poor surface properties, with the result that the surface of tubes made of such materials can generally be easily damaged, in particular when using clamps to close off such tubes. Most polyolefins likewise have problems withstanding the pressure of the liquid pressed through by a pump, and are thereby also not capable of transporting constant quantities of liquid.

In addition, most tubes made of polyolefins have a low tensile force resistance. The tensile force resistance correlates with the tensile modulus and the latter generally depends on the crystallinity of the polyolefin material. In contrast, for example in the case of PVC materials it depends on the quantity of added plasticizer.

Tubes made of polyolefin materials which have low tensile force resistance values have the disadvantage, in particular when used as pump tubes, that the diameter of the tube is deformed into an oval, with the result that the flow of the fluid through the tube is reduced or is not constant.

Moreover, it is also imperative, in order to achieve the required mechanical and physical properties in a pump tube application, to give the tube described in U.S. Pat. No. 6,187,400 the material properties necessary for use by ionizing irradiation during sterilization. However, the irradiation of polymers has disadvantages, as polymers can discolour and thus there is a lower market acceptance. Furthermore, safety requirements for a sterilization or material treatment by radiation ionization makes the production of such tubes undesirably laborious and costly.

EP 765740 B1 provides a PVC-free multilayer tube for medical purposes and a process for its production and use. The aim of this patent was to match different plastics layers in a multilayer tube material to one another such that at least one layer acts as a base layer and gives the tube material a sufficient thermal stability during the sterilization. Due to the material composition of the tube mentioned there, use as a pump tube is ruled out as, because of the small quantities of resistant polyolefins, the layers lying on the outside generally lack the required mechanical resistance, and the desired kink resistance is not achieved either as a result of the material mix. The tube mentioned there likewise tends to ovalize under pump tube conditions, because in particular the wall thicknesses used and the composition of the material mix rules out a use as a pump tube.

US 2003/0044555 describes a pump tube made of polybutadienes. This material also requires modification by ionizing irradiation. Furthermore, an annealing process must be carried out to increase the crystallinity of the material in order to achieve the desired properties. The process is likewise very laborious here also.

DE 44 46 896 describes impact-resistant, thermoplastically processable mixtures of elastomers and thermoplasts. From these mixtures composite materials are produced which are constructed from three layers of the polymeric mixtures, wherein the outer layers contain polyolefins and the middle layer thermoplastic elastomers.

The object was therefore to provide a tube in particular for use as a pump tube which is PVC-free and which has the physical and chemical properties required for application in a pump tube system. The object was therefore in particular to provide a pump tube which both has the elastic properties necessary for a pump tube and can, as a result of a high mechanical resistance, withstand the abrasive effects due to the conveyance roller on its outside. Furthermore, an ovalization of the tube during the pumping process should be avoided and a constant material quality achieved without a loss of delivery rate. In addition, the tube according to the invention should make it possible to avoid friction between sheets of material arranged alongside one another.

It was surprisingly found that a PVC-free tube which comprises three layers arranged one above another, wherein each of these layers contains a polyolefin and wherein a middle layer contains at least 60% of a thermoplastic elastomer, the loss factor of which relative to the temperature displays a maximum at a temperature of above −30° C., overcomes the disadvantages in the state of the art.

Within the framework of this invention, outer layer always means the layer furthest away from the centre of the tube cross-section and inner layer always means the layer closest to the centre of the tube cross-section. A middle layer always denotes a layer between the outer layer and the inner layer. There can be several middle layers.

It was furthermore shown that a PVC-free tube with three layers arranged one above another, wherein each of these layers contains a polyolefin and wherein a middle layer contains at least 60% of a thermoplastic elastomer, the loss factor of which relative to the temperature displays a maximum at a temperature of above −30° C., and which has a glass transition temperature T_{g} of above −35° C., displays an advantageous restoration loss.

It was surprisingly found that a PVC-free tube with three layers arranged one above another, wherein each of these layers contains a polyolefin and wherein a middle layer contains at least 60% of a thermoplastic elastomer, the loss factor of which relative to the temperature displays a maximum at a temperature of above −30° C. and which has a still measurable loss factor at service temperature, displays a lower tendency to buckle, which is also known as “kinking” to a person skilled in the art. The mechanical stress on a tube in a roller pump with a simultaneous periodic compressive stress due to the rollers is great and promotes the ovalization of the tube cross-section. The delivery rate decreases as a result.

This ovalization is much less when using the thermoplastic elastomer according to the invention.

Within the framework of this invention, a thermoplastic elastomer which has a still measurable loss factor at service temperature is a thermoplastic elastomer which has a loss factor of more than 0.01 at a temperature of 37° C. A tube according to the invention, which comprises a thermoplastic elastomer in a middle layer with this loss factor under the named conditions, has a restoration loss of less than 12%.

The loss factor, which is also known to a person skilled in the art as “tan delta”, is typically used as a variable to characterize dynamic mechanical behaviour. Within the framework of the present invention, dynamic mechanical analysis (DMA) according to the ISO 6721-7 method has been used. A value of 0.01 or a higher loss factor must be achieved according to the invention at 37° C. because dialysis tubes are used at this temperature and are to display a low conveyance rate loss at this temperature. Infusion tubes are used at room temperature. The thermoplastic elastomers are therefore preferably to have a loss factor of greater than 0.01 at a temperature of 20° C.

The restoration loss is here defined as the loss relative to the value of restoration force measured according to the method described in detail in the embodiment examples after 180 minutes. A detailed discussion of the restoration loss according to the invention or of the restoration value and the corresponding restoration force is to be found in the embodiment examples. This value thus gives the tube a degree of flexibility at a defined stability, which also means that the loss of delivery rate lies below the known value of 13% for PVC tubes. Furthermore, an ovalization of the tube according to the invention is avoided.

It was furthermore shown that a PVC-free tube with three layers arranged one above another, wherein each of these layers contains a polyolefin and wherein a middle layer contains at least 60% of a thermoplastic elastomer, the loss factor of which relative to the temperature displays a maximum at a temperature of above −30° C., and the loss modulus maximum G″ₘₐₓ of which lies above −35° C., shows an advantageous restoration loss.

Specifically the properties of the thermoplastic elastomer used are of particular importance for the properties of the tubes, as the middle layer containing the thermoplastic elastomer often displays a large layer thickness and the percentage by weight according to the invention of the thermoplastic elastomer in a middle layer constitutes the largest material portion at more than 60%.

The following correlations tend to be found: The pumping rate loss of the tube produced with the thermoplastic elastomer decreases if
the glass transition temperature T_{g} increases,
the loss modulus maximum G″ₘₐₓ shifts to higher temperatures,
the value of the loss factor is as high as possible at a service temperature of 37° C.,
the maximum loss factor lies at higher temperatures,
the compatibility of the thermoplastic elastomer vis-à-vis polypropylene increases.

According to the invention are thus all PVC-free tubes which comprise three layers arranged one above another, wherein each of these layers contains a polyolefin, and wherein a middle layer contains at least 60% of a thermoplastic elastomer and this thermoplastic elastomer as starting material has the following properties:
i) the loss factor relative to the temperature displays a maximum which lies above −30° C. in the case of the thermoplastic elastomers, or
ii) the thermoplastic elastomers have a glass transition temperature T_{g} of above −35° C., or
iii) the thermoplastic elastomer has a loss factor of more than 0.01 at a temperature of 37° C., or
iv) the loss modulus G″ relative to the temperature has a maximum which lies above −35° C. in the case of the thermoplastic elastomers according to the invention.

It was shown that thermoplastic elastomers with one of the properties named under i to iv are particularly well suited to the production of the tubes according to the invention, with the result that the tubes produced with these thermoplastic elastomers display excellent low restoration and pumping rate losses.

By “polyolefins” are meant here polymers which are constructed from carbon and hydrogen atoms and can contain single and multiple bonds. Polyolefins usually do not contain aromatic units. For a definition of polyolefins, reference is made to Oberbach, Baur, Brinkmann, Schmachtenberg “Saechtling-Kunststofftaschenbuch” Chap. 6.1, 29^{th} edition, Carl-Hanser-Verlag.

Unless otherwise indicated below, quoted percentages usually relate to wt. −%.

Because of the high thermoplastic elastomer content, the middle layer containing the thermoplastic elastomer in the three-layer arrangement according to the invention gives the tube according to the invention the desired properties in respect of kink resistance, restoration capacity and delivery rate. The use of a high thermoplastic elastomer content is the reason for the low restoration loss and the low pumping rate loss. This surprisingly leads to the result that the loss of the delivery rate lies in the acceptable range of less than 13%, with the result that the tube can be used advantageously in particular for conveying blood in haemodialysis.

At least 20% of a polyolefin is contained in the layers (inner and outer layer) enclosing the middle layer. As a result of this content of a mechanically stable polyolefin, the outer and inner layers act essentially as a supporting layer and give the tube the required stability, in particular at the temperatures of 121° C. and higher customary in heat sterilization which cause the middle layer to soften due to the high thermoplastic elastomer content. The high polyolefin content also ensures that both the outer and the inner layers resist abrasive effects, for example during a pumping process.

The polyolefin content is preferably different in the inner and outer layers. It is preferred in particular that the polyolefin content in the inner layer is higher than in the outer layer. The result is advantageously that noise occurring due to the rubbing of the inner layers during an occlusion phase can be avoided. Furthermore, a tendency to block is thereby ruled out, which means that the tube opens automatically immediately after the occlusion and the inner layers of the tube do not adhere to each other. Moreover, the high polyolefin content in the inner layer guarantees a largely friction-free use, with the result that no friction residues can enter the biological liquid transported in the tube and a contamination can thus be avoided.

The high polyolefin content of more than 20% in the outer layer gives this layer a sufficiently large resistance to an external mechanical stress, for example due to the roller pump.

In a preferred embodiment of the invention, the polyolefin is selected from polymers of ethylene, propylene, butadiene, isoprene, copolymers and terpolymers thereof, and also polymer blends. These are polymers customary in the trade which can be produced cost-effectively, and are available and can be easily processed.

The thermoplastic polymer consists of aromatic and polyolefinic units and is preferably selected from the group consisting of styrene-ethylene-butadiene-styrene block copolymers (SEBS), styrene-butadiene-styrene copolymers (SBS), styrene-ethylene-propylene-styrene block copolymers (SEPS), styrene-ethylene-butadiene copolymers (SEB) and also styrene-isoprene-styrene block copolymers (SIS) and their mixtures (blends). These thermoplasts are rubber-elastic chemically uncross-linked polymers. They have the advantage that they remain dimensionally stable during heat sterilization, but at the same flow freely under shearing, such as e.g. during extrusion. The materials are completely amorphous, and consequently there can be no material influences due to crystallization processes such as can occur in the case of partly crystalline polymers after extrusion. These thermoplastic polymers can be particularly well mixed and processed with polyolefins to form blends and deliver the microphase structure required for the applications described above and below of the tube according to the invention, which has a determining influence on the necessary mechanical properties of the tube according to the invention.

In the particularly preferred embodiments of the invention, the thermoplastic polymer is SEBS or SIS.

The layer thickness of the outer layer is 30-250 μm, preferably 40-100 μm, more preferably 55-80 μm. The high polyolefin content in the outer layer makes it possible for the latter to be kept very thin compared with other outer layers in tubes from the state of the art which consist of several sheets of material, but nevertheless have a high stability. It is understood that further additional layers can also be applied to this outer layer if necessary. Likewise, further sheets of material can also be arranged if necessary between the individual layers of the three-layer system according to the invention. However, the basic sequence of three layers with the features according to the invention is essential for the present invention.

The layer thickness of the middle layer containing the thermoplastic elastomer is 400-3000 μm, preferably 1000-3000 μm and more preferably 1800-2000 μm. This layer thickness of the middle layer containing the thermoplastic elastomer in combination with the chosen thermoplastic elastomer mixture makes possible here the optimum compromise with regard to kink resistance and restoration capacity.

According to the invention, the ratio of the layer thickness of outer or inner layer to the middle layer containing the thermoplastic elastomer is between 1 to 8 and 1 to 25. Thus it is ensured that different sizes of tube, i.e. tubes with different internal diameters, can be provided, wherein the tubes have the desired properties in respect of kink resistance and restoration capacity.

The layer thickness of the inner layer is preferably 30-250 μm, quite particularly preferably 40-100 μm, still more preferably 55-80 μm. Here also, as a result of the use of a high polyolefin content, the thickness of the inner layer can be chosen relatively small without abrasion losses occurring or the mechanical supporting action of the inner layer being impaired.

Depending on use and area of application, the total wall thickness of the tube is 0.45-3.5 mm, preferably 2-2.2 mm in combination with an internal diameter of 3-28 mm, preferably 3-15 mm. The outer diameter of the tube is 4-35 mm, preferably 12-13 mm.

In a further preferred embodiment, the outer layer contains a compound which can absorb electromagnetic radiation and convert it into heat energy. Thus for example polypropylene connectors can be used particularly easily in a tube system because a secure weld between tube and connector can be made by laser. Such laser welding techniques are known for example from DE 10245355 A1.

Examples of such compounds are organic dyes or UV absorbers which absorb the laser light in the wavelength range of the laser used. Likewise inorganic compounds such as calcium silicate or iron oxide can also be used provided the colouring has no undesired effects.

Suitable compounds are disclosed i.a. in ANTEC 2000, Conference Proceedings (Jones, I. A. and Tayler, N. S., Use of infrared Dyes for Transmission Laser Welding of Plastics, pp. 1166-1169) and in WO 02/00144 A1.

The problem of the present invention is further solved by a tube system comprising a plurality of tubes according to the invention or tube segments according to the invention. The tube system preferably comprises at least two different tubes or tube segments which are connected via a connector. The connector is preferably composed of a polyolefin, in particular polypropylene.

Such tubes and tube systems according to the invention are preferably used as pump tubes in an extracorporeal blood circulation, in enteral feeding, infusion or transfusion.

The invention is described in more detail with reference to the diagrams below and with reference to embodiment examples, which are not, however, to be considered limiting.

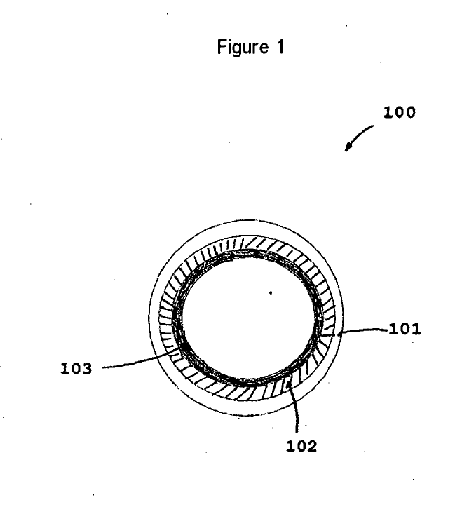

FIG. 1 shows a cross-section view through a tube according to the invention.

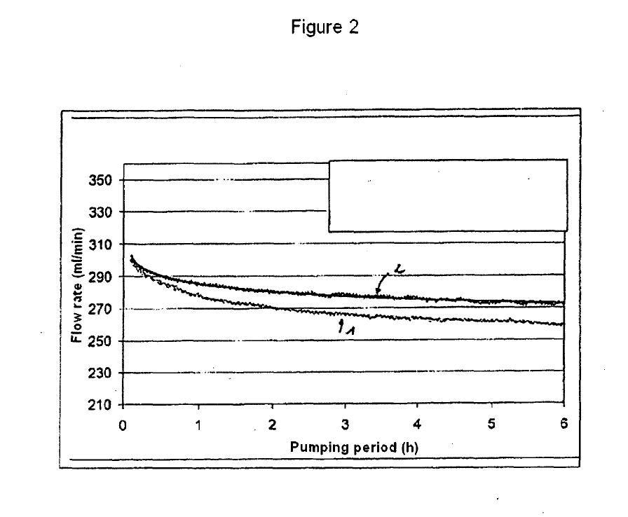

FIG. 2 shows the time-related conveyance rate of pump tubes according to the invention compared with conventional PVC pump tubes, wherein the conveyance rate is plotted against the pumping period.

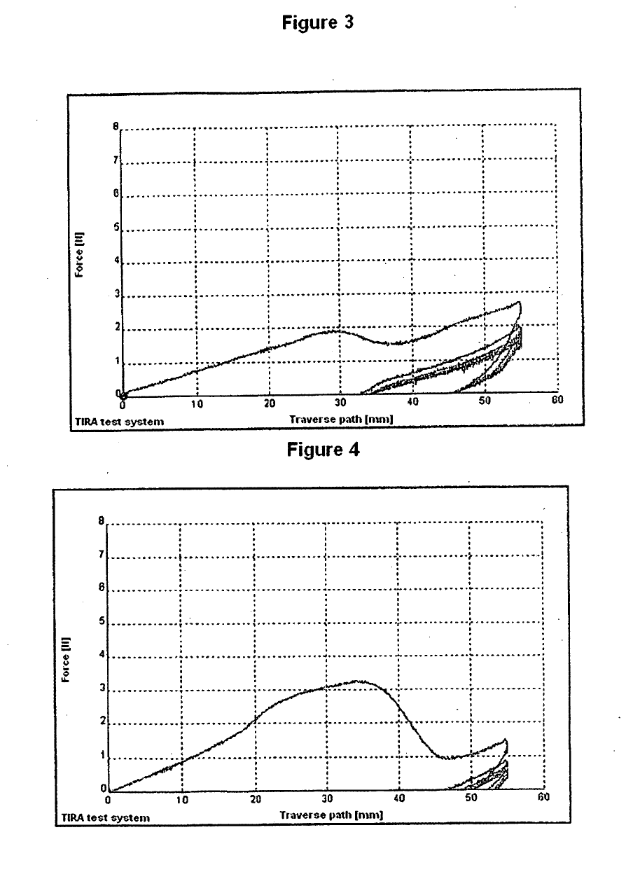

FIG. 3 shows the kink resistance of a conventional PVC tube.

FIG. 4 shows the kink resistance of a tube according to the invention.

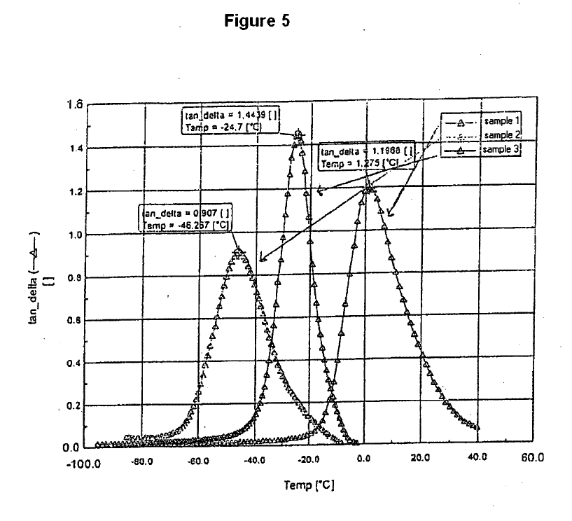

FIG. 5 shows the loss factor tan delta relative to the temperature of three samples.

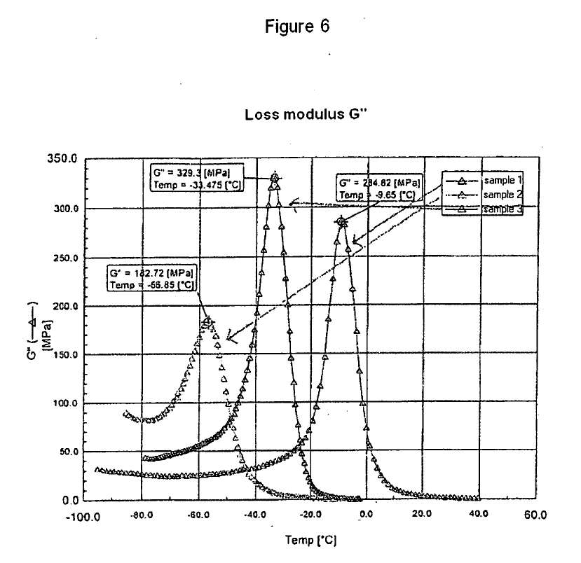

FIG. 6 shows the loss modulus G″ relative to the temperature of three samples.

FIG. 1 shows a cross-section view through a tube 100 according to the invention. The tube 100 consists of three layers 103, 102 and 101 arranged one above another. The outer layer 101 consists of a mixture of 55% SEBS (Tuftec H1221, Asahi), 5% SEBS (Septon 4077, Kuraray), 35% PP-R (RB 501 BF, Borealis) and 200 ppm of an amide wax (Crodamide ER). Naturally, suitable polyethylene or polypropylene mixed copolymers and blends etc. can also be used instead of the polypropylene used. The inner layer 103 consists of 60% PP-R (RD 208 BF, Borealis) and 40% SEBS (Tuftec H1221, Asahi). Naturally, the polypropylene content in the inner and outer layer can also be chosen the same, but it is preferred, as already stated above, that the polypropylene content or the polyolefin content in the inner and outer layer is different, quite particularly preferred as in the present case that the polypropylene content in the inner layer is greater than in the outer layer in order to avoid abrasion losses.

The middle layer consisting of 80% SIS (Hybrar 7125 F, Kuraray) and 20% PP (Borsoft SC220, Borealis) is arranged between layers 103 and 101. Naturally, a correspondingly different thermoplastic elastomer such as for example SEBS or SEPS can also be used instead of SIS.

### General Test Procedure to Determine the Conveyance Rate Loss:

The following test set-up was chosen to determine the conveyance rate loss of the pump tubes according to the invention examined below:

The pump tube segment was inserted in a roller pump which is normally used in haemodialysis. A water-glycerol mixture kept at 37° C. is sucked in using the pump. The mixture had a similar viscosity to human blood in order to compare the measurement results with the conditions to be expected in practice. The delivery rate was kept constant and the delivered volume determined in ml/min. The conveyance rate loss was determined in % after 6 hours' conveyance.

A PVC pump tube (8.0 mm internal diameter×2.1 mm wall thickness) from Sis-Ter s.p.a. (product number 6961941) was used as material (material name: Evicom AM561/65SH).

A tube roller pump customary in the trade was used to determine the conveyance rate loss, wherein a rotor incursion or an occlusion was measured over a circle arc segment of approx. 270°. The rotor forces correspond to the dimensions of the roller pump of the model 4008 Fresenius dialysis machine. In the design of the rotor, cylindrical rollers were chosen and the pump tube coupling was safeguarded by feeding via a pump tube adapter. A through flow meter for continuous recording of the effective flow rates over a period of six hours was integrated. The fluid is sucked in and returned via cannulas with a diameter of 1.5 mm.

The stress conditions which occur in practice in haemodialysis systems were simulated by setting the following parameters:

| | |
|---|---|
| Throughflow: | 300 ml/min |
| Fluid temperature: | 37° C. (corresponds to the temperature |
| | of human blood) |
| Fluid viscosity: | 3.6 mPa * s (corresponds to the viscosity |
| | of human blood) |
| Duration: | 6 h (corresponds to the maximum duration |
| | of standard haemodialysis treatments) |
| Pressure conditions | approx. −390 mm Hg/+170 mm Hg. |
| (before and after | |
| the pump): | |

All the tested tubes were steam-sterilized at 121° C. before use.

### EXAMPLE 1

The tubes according to the invention of Examples 1-3 were prepared by coextrusion and introduced after extrusion into a water bath kept at 20° C. and annealed. A negative pressure was simultaneously applied in a vacuum calibration in the extruded tube in order to keep the tube measurements constant after extrusion. The layer thicknesses of the individual layers were 60 μm in each case for the outer and inner layers and 1980 μm for the middle layer, with the result that the tube according to Examples 1-3 had a total wall thickness of 2.1 mm. The internal diameter was in each case 8 mm.

A three-layer tube according to the invention as shown in FIG. 1 was produced from the following materials:
1. The outer layer consisted of a mixture (blend) of:
   55% SEBS (Tuftec H1221, Asahi)
   5% SEBS (Septon 4077, Kuraray)
   35% PP-R (RB 501 BF, Borealis)
   200 ppm (Crodamide ER amide wax).
2. The middle layer consisted of a mixture of:
   85% SEBS (Tuftec 1221, Asahi)
   15% PP-R (RD 204 CF).
3. The inner layer consisted of a mixture of:
   60% PP-R (RD 208 BF, Borealis)
   40% SEBS (Tuftec H1221, Asahi).

After 6 hours' conveyance in a roller pump at a conveyance rate of 300 ml/min, the conveyance rate loss was 21.9%.

### EXAMPLE 2

A tube with the following structure was produced:
1. The outer layer consisted of a mixture of:
   55% SEBS (Tuftec H1221, Asahi)
   5% SEBS (Septon 4077, Kuraray)
   35% PP-R (RB 501 BF, Borealis)
   200 ppm (Crodamide ER amide wax).
2. The middle layer consisted of a mixture of:
   85% SIS (Hybrar 7125 F, Kuraray)
   15% PP-R (RD 204 CF).
3. The inner layer consisted of a mixture of:
   60% PP-R (RD 208 BF, Borealis)
   40% SEBS (Tuftec H1221, Asahi).

The conveyance rate loss was 13.6% after 6 hours and a conveyance rate of 300 ml/min.

### EXAMPLE 3

A further tube composed of the following materials was produced:
1. The outer layer consisted of a mixture of:
   55% SEBS (Tuftec H1221, Asahi)
   5% SEBS (Septon 4 077, Kuraray)
   35% PP-R (RB 501 BF, Borealis)
   200 ppm (Crodamide ER amide wax).
2. The middle layer consisted of a mixture of:
   80% SIS (Hybrar 7125 F, Kuraray)
   20% PP (Borsoft SC220, Borealis).
   3. The inner layer consisted of a mixture of:
   60% PP-R (RD 208 BF, Borealis)
   40% SEBS (Tuftec H1221, Asahi).

The conveyance rate loss was 9.3% after 6 hours and a conveyance rate of 300 ml/min.

FIG. 2 shows the time-related conveyance rate of a pump tube according to the invention compared with a PVC pump tube of the state of the art. The flow rate is shown in ml/min relative to the pumping period. The motor output of the pump is kept constant for the duration of the pumping period.

Curve 1 shown as a dotted line shows the time-related conveyance rate of a PVC tube of the state of the art (internal diameter: 8 mm, wall thickness; 2.1 mm).

The solid curve 2 shows the corresponding measurement results of a PVC-free pump tube according to the invention according to Example 3 (internal diameter: 8 mm, wall thickness: 2.1 mm).

FIG. 2 shows that the conveyance rates for both pump tubes decreases as the conveyance duration increases. However, the decrease in the conveyance rate in the PVC-free tube according to the invention (curve 2) is not as pronounced as with the PVC tube (curve 1).

The kink resistance of tubes according to the invention was studied further by a TIRA tensile testing machine. The respective tube or tube segment was attached by its ends to two clamping jaws. The distance between the clamping jaws was 60 mm. The inserted tube was 240 mm long. It lay curved between the test clamping jaws. The test clamping jaws were moved towards each other at a rate of 240 mm/min. The force with which the tube opposes the clamping jaws was measured. In addition, the reduction in the distance between the clamping jaws, the so-called transfer path, was measured.

FIGS. 3 and 4 show that the force initially increases up to a maximum depending on the transfer path. This maximum corresponds to the buckling of the tube. As a result of the buckling, the tube loses its tension over its whole length and can oppose the test clamping jaws with only a low force. After kinking, therefore, a decrease in force was observed as the transfer path increases. If the force path course is followed further with reference to FIGS. 3 and 4, a fresh rise in the curve is observed. Here, the tube is already compressed in the test machine to the point where a fresh stress develops against the test clamping jaws.

It is desirable for use as a pump tube that buckling occurs only after the greatest possible transfer path has been covered and that the drop in force after kinking is not too great. Taking the example of a PVC pump tube customary in the trade (FIG. 3), it can be seen that no full buckling has taken place on the transfer path investigated. Only a slight buckling was observed for a transfer path of approx. 30 mm. The reason for this is the molecular structure of the polyvinyl chloride which is present in a partly solvated state as a result of the plasticizer used. The polymer chains of the PVC therefore display a degree of mobility and can partly compensate for the stress building up in the test piece by a slippage of the polymer chains. In contrast, the tube according to the invention according to Example 2 (FIG. 4) starts to buckle only after approx. 35 mm.

The restoration force or the restoration capacity according to the invention was likewise measured as follows with a TIRA tensile testing machine: For this, the tube was placed between the test clamping jaws which were then pushed together by 7 mm. The force with which the tube opposed the clamping jaws was then measured. To record the decrease in restoration forces during a pumping process, the tube was removed several times from the roller pump, after defined periods of pump use which are listed in Table 1, and surveyed in the testing machine.

TABLE 1
Restoration capacity of a PVC tube of the state of the art

| | Force | Loss relative to the |
|---|---|---|
| PVC | [N] | 5-min value: |
| Value after 5 min: | 25.96 | 0.00% |
| Value after 10 min: | 25.39 | −2.20% |
| Value after 15 min: | 25.1 | −3.31% |
| Value after 30 min: | 24.48 | −5.70% |
| Value after 60 min: | 23.9 | −7.94% |
| Value after 120 min: | 23.49 | −9.51% |
| Value after 180 min: | 23.09 | −11.06% |

TABLE 2
Restoration capacity of tubes according to the invention

| | Force | Loss relative to the |
|---|---|---|
| | [N] | 5-min value: |
| Example 1 | | |
| Value after 5 min: | 37.01 | 0.00% |
| Value after 10 min: | 36.19 | −2.2% |
| Value after 15 min: | 35.71 | −3.5% |
| Value after 30 min: | 34.92 | −5.6% |
| Value after 60 min: | 34.14 | −7.8% |
| Value after 120 min: | 33.42 | −9.7% |
| Value after 180 min: | 32.82 | −11.3% |
| Example 2 | | |
| Value after 5 min | 38.59 | 0.00% |
| Value after 10 min: | 37.69 | −2.3% |
| Value after 15 min: | 37.11 | −3.8% |
| Value after 30 min | 36.06 | −6.6% |
| Value after 60 min | 35.27 | −8.6% |
| Value after 120 min | 34.71 | −10.1% |
| Value after 180 min: | 34.16 | −11.5% |
| Example 3 | | |
| Value after 5 min | 52.07 | 0.00% |
| Value after 10 min: | 50.78 | −2.5% |
| Value after 15 min: | 50.04 | −3.9% |
| Value after 30 min | 48.8 | −6.3% |
| Value after 60 min | 47.77 | −8.3% |
| Value after 120 min | 46.88 | −10.0% |
| Value after 180 min: | 46.09 | −11.5% |

Table 2 shows that, although the tubes according to the invention displayed a somewhat greater loss of restoration capacity overall than a PVC tube (Table 1), the differences are only slightly above the value of 11%, to be regarded as optimal.

FIG. 5 shows the loss factor tan delta relative to the temperature of the three commercially available samples “Hybrar 7125 F” (sample 1), “Tuftec 1062” (sample 2) and “Tuftec 1221” (sample 3). The measurement of the loss factor was carried out on all samples in accordance with ISO 6721-7. Test pieces composed of the three block copolymers were produced by pressing the granular sample material at a temperature of 200° C. into sheets approx. 4 mm thick. Test pieces measuring 80 mm×10 mm×plate thickness were produced from the pressed sheets. A Rheometric Scientific “Torsion head” DMA measuring head was used as testing apparatus.

The test conditions were as follows:
type of stress: forced torsional vibration
frequency: 1 Hz
temperature: −100° C. to room temperature or 40° C.
heating rate: 1 K/min
flushing gas: dry air

The maxima of the loss factor of the samples are shown in FIG. 5.

The Hybrar sample, which is commercially available from Kuraray, is a styrene/isoprene/styrene block copolymer (SIS block copolymers). The Tuftec samples, which are commercially available from Asahi Kasei, are SEBS-type styrene block copolymers.

The Hybrar sample (sample 1) was used in the abovementioned Examples 2 and 3 of this application. A reduction of the pumping rate loss is achieved when the thermoplastic elastomer is used in the middle layer, containing the thermoplastic elastomer, of the tube. In Examples 2 and 3, the pumping rate loss is 13.6% and 9.3% respectively.

Tuftec 1062 (sample 2) was not used in any of the examples listed. A pump tube which was produced using this material had a pumping rate loss which was greater than 20%. Thus this SEBS type is not suitable to produce tubes according to the invention which contain this thermoplastic elastomer in the middle layer of the tube. As FIG. 5 shows, a measurable loss factor of more than 0.01 can be achieved only at temperatures of approx. −10° C.

Tuftec 1221 (sample 3) was used in Example 1. A measurable loss factor of more than 0.01 is achieved at a similarly low temperature as with Tuftec 1062 (approx. −5° C.). As already mentioned above, the conveyance rate loss after 6 hours' conveyance in a roller pump is 21.9% at a conveyance rate of 300 ml/min.

Using the sample which displays a still measurable loss factor at 37° C., a tube according to the invention is obtained which displays an advantageous restoration loss.

FIG. 6 shows the loss modulus G″ relative to the temperature. It was shown that the loss modulus maximum for sample 1 occurs at the highest temperature (−9.6° C.). The loss modulus maxima of samples 2 and 3 are in some cases markedly lower at −56.85° C. and −33.48° C.

Table 3 summarizes the properties of the thermoplastic elastomers studied by way of example:

TABLE 3
Properties of the studied thermoplastic elastomers

| | | | T_{g} | | | |
|---|---|---|---|---|---|---|
| | | Ex. | [° C.]/ | G″ ₘₐₓ | Loss factor | Loss factor max. |
| Material: | Sample | number | DSC | [° C.] | at 37° C. | [° C.] |
| SIS Hybrar | 1 | 2 and 3 | −9.2 | −9.6 | 0.083 | 1.2 |
| 7125 F | | | | | | |
| SEBS Tuftec | 2 | — | −54.7 | −56.8 | — | −46.3 |
| 1062 | | | | | | |
| SEBS Tuftec | 3 | 1 | −33.5 | −33.5 | — | −24.7 |
| 1221 | | | | | | |

1. PVC-free tube (100) comprising three layers (101, 102, 103) arranged one above another, wherein each of these layers contains a polyolefin, characterized in that a middle layer (102) contains at least 60% of a thermoplastic elastomer, the loss factor of which relative to the temperature displays a maximum at a temperature of above −30° C.

2. Tube according to claim 1, in which the thermoplastic elastomer has a glass transition temperature T_{g} of above −35° C.

3. Tube according to claim 1, in which the thermoplastic elastomer has a loss factor of more than 0.01 at a temperature of 37° C.

4. Tube according to claim 1, in which the thermoplastic elastomer has a loss modulus maximum G″ₘₐₓ of above −35° C.

5. Tube according to claim 1, characterized in that at least 20% of a polyolefin are contained in the outer and inner layers (101, 103).

6. Tube according to claim 1, characterized in that the polyolefin content in layers (101) and (103) is different.

7. Tube according to claim 6, characterized in that the polyolefin content in the inner layer (103) is greater than in the outer layer (101).

8. Tube according to claim 1, characterized in that the polyolefin is selected from polyethylene, polypropylene, their copolymers, terpolymers and mixtures thereof.

9. Tube according to claim 8, characterized in that the thermoplastic polymer is selected from the group consisting of SEBS, SBS, SEPS, SEB, SIS and mixtures thereof.

10. Tube according to claim 9, characterized in that the thermoplastic polymer is SEBS or SIS.

11. Tube according to claim 1, characterized in that the layer thickness of the outer layer (101) is 30-250 μm, preferably 40-100 μm.

12. Tube according to claim 1, characterized in that the middle layer containing the thermoplastic elastomer has a layer thickness of 400-3000 μm, preferably 1000-3000 μm and more preferably 1800-2000 μm.

13. Tube according to claim 1, characterized in that the layer thickness of the inner layer (103) is 30-250 μm, preferably 40-100 μm.

14. Tube according to claim 1, characterized in that the ratio of the layer thicknesses of the outer layer to the middle layer containing the thermoplastic elastomer is between 1 to 8 and 1 to 25.

15. Tube according to claim 1, characterized in that the ratio of the layer thicknesses of the inner layer to the middle layer containing the thermoplastic elastomer is between 1 to 8 and 1 to 25.

16. Tube according to claim 1, characterized in that the total wall thickness of the tube is 0.45-3.5 mm, preferably 2-2.2 mm.

17. Tube according to claim 1, characterized in that the internal diameter of the tube is 3-28 mm, preferably 3-15 mm.

18. Tube according to claim 1, characterized in that the outer diameter of the tube is 4-35 mm, preferably 12-13 mm.

19. Tube according to claim 1, characterized in that the outer layer (101) also contains a compound which can absorb electromagnetic radiation and convert it into heat energy.

20. Tube system comprising a plurality of tubes according to claim 1.

21. Tube system according to claim 20, characterized in that at least two tubes are connected via a connector which is composed of a polyolefin.

22. Tube system according to claim 21, characterized in that the connector is composed of polypropylene.

23. Use of a tube according to claim 1 in an extracorporeal blood circulation.

24. A method of providing extracorporeal blood circulation wherein blood is circulated in a tube according to claim 1.

25. A method of providing extracorporeal blood circulation wherein blood is circulated in a tube system according to claim 20.
